(19) United States
(12) Patent Application Publication
(10) Pub. No.: US 2026/0102676 A1
(43) Pub. Date: Apr. 16, 2026

(54) SYSTEM FOR PROVIDING AI CADDIE SERVICE AND OPERATING METHOD THEREOF

(71) Applicants: Sang Hyun LEE, Seoul (KR); MATE MOBILITY CO., LTD., Seoul (KR)

(72) Inventors: Sang Hyun LEE, Seoul (KR); Seung Joon LEE, Seoul (KR); Jae Young CHOI, Gyeonggi-do (KR); Hae Young CHOI, Gyeonggi-do (KR); Jeong Beom SEO, Seoul (KR); Il Do KIM, Seoul (KR)

(21) Appl. No.: 19/346,563

(22) Filed: Oct. 1, 2025

(30) Foreign Application Priority Data
Oct. 10, 2024 (KR) 10-2024-0137709

(51) Int. Cl. A63B 69/36; A63B 71/06

(52) U.S. Cl. CPC A63B 69/3605; A63B 71/0622; A63B 2220/10

(57) ABSTRACT

An artificial intelligence (AI) caddie service provision system includes a terminal mounted on a single golf cart in which a player rides and receiving information on a club used for a shot after the player takes the shot and moves the single golf cart to a new location of a ball, and a server learning distance information of the club used for the shot based on location information of the terminal at the new location of the ball and the information on the club used for the shot, and then providing the terminal with strategy information for a next shot to be taken from the new location of the ball.

### CROSS-REFERENCE TO RELATED APPLICATION

The present application claims priority under 35 U.S.C. § 119(a) to Korean patent application number 10-2024-0137709, filed on Oct. 10, 2024, the entire disclosure of which is incorporated herein by reference.

### BACKGROUND

### 1. Technical Field

Various embodiments of the present disclosure generally relate to an artificial intelligence (AI) caddie service provision device and an operating method thereof, and more particularly, to an AI-based caddie service provision terminal device which assists golf players during their golf rounds and a method of operating the AI-based caddie service provision terminal device.

### 2. Related Art

A caddie is not merely a porter who carries a golf bag and follows a player around a course, but they play a crucial role by providing various advice to the player, considering factors, such as the course terrain, wind direction, and the player's condition. Professional golfers hire their own personal caddies, but amateur golfers can pay a fee (caddie fee) to receive services from caddies employed by a golf course, similar to renting a multi-passenger golf cart. However, when multiple players share the assistance of one caddie, it may be difficult for each player to receive optimized advice tailored to their specific needs.

The use of single-rider carts and unmanned carts has been increasing at golf courses recently. Accordingly, there are opinions that caddies are unnecessary, but the presence or absence of a caddie is a crucial factor directly affecting a player's performance. Therefore, even when using single-rider or unmanned carts, players still need receive caddie services.

### Prior Art Reference

### Patent Documents

(Patent Document 0001) Korean Laid-Open Patent Publication No. 2024-91621 (Published on Jun. 21, 2024)
(Patent Document 0002) Korean U.S. Pat. No. 2,012,963 (Issued on Aug. 21, 2019)

### SUMMARY

Various embodiments of the present disclosure are directed to an artificial intelligence (AI) caddie service provision system and an operating method thereof.

An artificial intelligence (AI) caddie service provision system according to an embodiment of the present disclosure may include a terminal mounted on a single golf cart in which a player rides and receiving information on a club used for a shot after the player takes the shot and moves the single golf cart to a new location of a ball, and a server learning distance information of the club used for the shot based on location information of the terminal at the new location of the ball and the information on the club used for the shot, and then providing the terminal with strategy information for a next shot to be taken from the new location of the ball.

In an embodiment, the terminal may include a real-time kinematic (RTK) module acquiring the location information of the terminal using RTK positioning, a wireless communication module connected to a wireless communication network to perform data communication between the terminal and the server, and a controller controlling the wireless communication module to provide the location information of the terminal to the server.

In an embodiment, the AI caddie service provision system may further include a database storing player record information which includes previous play information of the player and information on a golf course on which the player plays a round.

In an embodiment, the server may include a shot result information generation module receiving the location information of the terminal and the information on the club used for the shot from the terminal, and generating shot result information including club-specific distance information and shot trajectory information of the player, a play learning module acquiring the player record information from the database and updating the player record information based on the shot result information, and a course strategy information generation module generating course strategy information for the next shot based on the updated player record information.

In an embodiment, the shot result information generation module may determine information on a distance from a location of the terminal from which the player took the shot to the location of the terminal at the new location of the ball as the distance information of the club used for the shot.

In an embodiment, the player record information may include club-specific distance information, shot trajectory information, and shot accuracy information learned from past play results of the player.

In an embodiment, the learned club-specific distance information, shot trajectory information, and shot accuracy information included in the player record information may further include information based on temperature or humidity.

In an embodiment, the course strategy information generation module may generate stable course strategy information and aggressive course strategy information by using results of learning past playing tendencies of the player.

In an embodiment, the course strategy information may include directional information, distance information, and an expected success rate per club for the next shot.

In an embodiment, the single golf cart may be capable of directly entering a fairway included in a golf course and moving to a location where the ball of the player is located, except for a pre-set restricted area.

### BRIEF DESCRIPTION OF THE DRAWINGS

FIG. 1 is a diagram illustrating a golf playing method using a single golf cart;

FIG. 2 is a diagram illustrating an artificial intelligence (AI) caddie service provision system according to an embodiment of the present disclosure;

FIG. 3 is a diagram illustrating a configuration of a terminal of FIG. 2;

FIG. 4 is a diagram illustrating a configuration of a server of FIG. 2;

FIG. 5 is a diagram illustrating a method by which an AI caddie service provision system of the present disclosure learns club-specific distance information of a player;

FIG. 6 is a diagram illustrating receiving information about clubs to be used, through an input unit of FIG. 3;

FIG. 7 is a diagram illustrating receiving information about a club used, through the input unit of FIG. 3;

FIG. 8 is a diagram illustrating a method by which an AI caddie service provision system of the present disclosure provides course strategy information; and

FIG. 9 is a diagram illustrating an example of an AI caddie service provided by an AI caddie service provision system of the present disclosure.

### DETAILED DESCRIPTION

Specific structural or functional descriptions disclosed herein are merely illustrative for the purpose of describing embodiments according to the concept of the present disclosure. Embodiments according to the concept of the present disclosure may be implemented in various forms and should not be construed as being limited to the specific embodiments set forth herein.

Hereinafter, embodiments are described with reference to the accompanying drawings in order for those skilled in the art to be able to implement the technical spirit of the present disclosure.

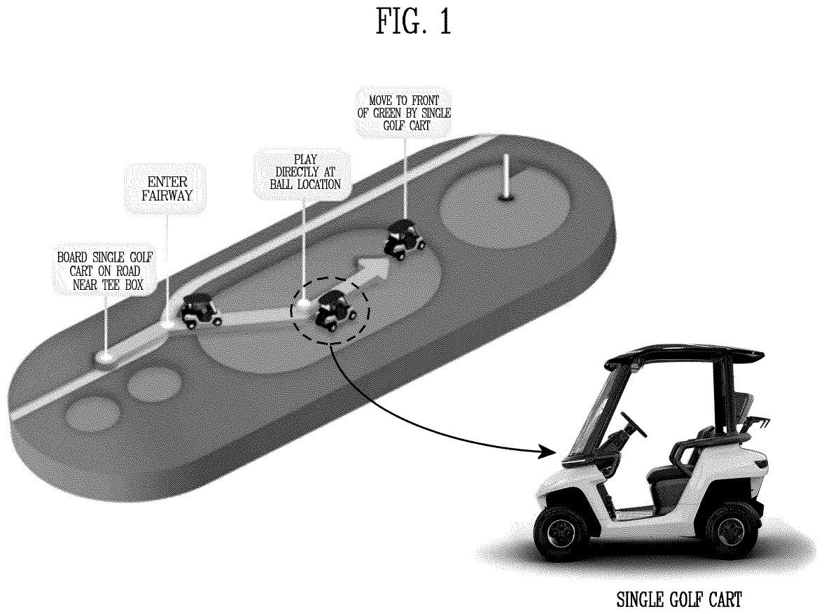

FIG. 1 is a diagram illustrating a golf playing method using a single golf cart.

Referring to FIG. 1, a single golf cart may be a cart which allows each player participating in a round to ride individually and drive their own cart. That is, when four players are playing, up to four single golf carts may be used. Because each single golf cart may carry equipment of each player, players may enjoy golf play using their own single golf cart.

After hitting a tee shot from a tee box, the player may drive their own single golf cart to a location where their ball has landed. Unlike the conventional practice where three or more players ride in one cart and move only along pre-set cart paths, single golf carts may directly enter a fairway.

Specifically, the single golf cart may directly enter a fairway included in a golf course, except for pre-set restricted areas, and move to the location of the ball of the player. Therefore, using the single golf cart can minimize or reduce a distance a player must walk, shortening a golf playing time. In an embodiment, restricted areas for single golf carts may include the rough, bunker, penalty area, and green (including apron). Here, the term “penalty area” may refer to areas where shots cannot be taken, such as lakes, streams, or drainage ditches. As an exception, in the case of a water hazard, the player may either enter the water to make a shot directly or take a one-stroke penalty and drop the ball at a new location to play the next shot. Rules for playing from penalty areas may vary depending on local rules established by each golf course.

Once the ball of the player is on the green, the player may use their single golf cart to move to the green entrance. Because the player drives the single golf cart themselves, they may move directly to an area they wish to play in and continue playing, provided it is not a restricted area.

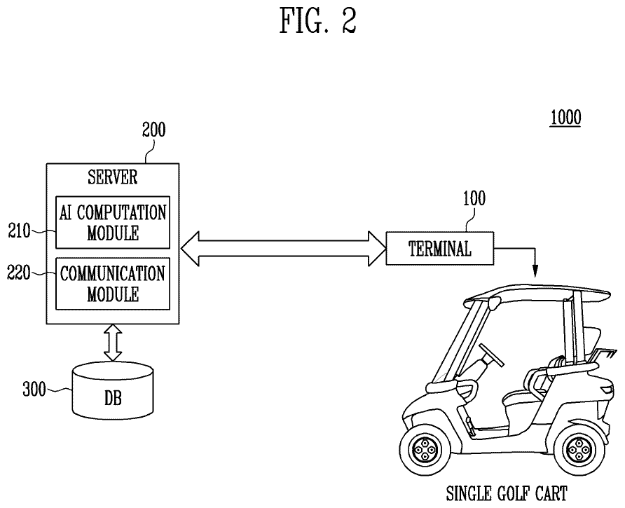

FIG. 2 is a diagram illustrating an artificial intelligence (AI) caddie service provision system according to an embodiment of the present disclosure. In the present disclosure, the term “AI caddie service provision system”refers to a system for providing an AI caddie service.

Referring to FIG. 2, the AI caddie service provision system may include a terminal 100, a server 200, and a database (DB) 300. The AI caddie service provision system is network-based, where the network refers to a connection structure enabling information exchange between respective nodes such as terminals 100 and servers 200.

The terminal 100 may be mounted on the single golf cart as described with reference to FIG. 1. The terminal 100 may be a tablet PC, but is not limited thereto, and may be implemented as various forms of electronic devices such as a smartphone, a laptop computer, or an in-vehicle infotainment system.

In an embodiment, the terminal 100 may be controlled by input from a player using the terminal 100 and may communicate with the server 200 via wireless communication. The terminal 100 is mounted on the single golf cart, enabling an environment where the player may freely use the terminal 100 during golf play. The terminal 100 may perform data communication with the server 200 using a wireless communication system.

The terminal 100 may provide various data related to playing golf to the server 200 and receive data concerning the AI caddie service from server 200. The terminal 100 may provide the AI caddie service to the player based on the data received from the server 200.

The server 200 may be provided at a specific location within the golf course or at a remote location outside the golf course. The server 200 may perform data communication with the terminal 100 using a wireless communication system. The wireless communication system used by the terminal 100 and the server 200 may include a 3rd Generation Partnership Project (3GPP) network, a Long Term Evolution (LTE) network, a 5th Generation (5G) Mobile Telecommunication, a World Interoperability for Microwave Access (WiMAX) network, the Internet, a Local Area Network (LAN), a Wireless Local Area Network (Wireless LAN), a Wide Area Network (WAN), a Personal Area Network (PAN), a Bluetooth network, a satellite broadcasting network, an analog broadcasting network, a Digital Multimedia Broadcasting (DMB) network, Wi-Fi, or the like, but is not limited thereto.

The server 200 may include an AI computation module 210 and a communication module 220.

The AI computation module 210 may perform various computations to generate data used for providing the AI caddie service, using data stored in the DB 300 and data provided from the terminal 100. The AI computation module 210 may generate data used for providing the AI caddie service using various algorithms such as deep learning, machine learning, and a neural network circuit utilizing big data. The AI computation module 210 may provide data used for providing the AI caddie service to the terminal 100 using the communication module 220.

The communication module 220 may perform data communication with the terminal 100 and the DB 300.

The DB 300 may store player record information. The player record information may include club-specific distance information, shot trajectory information, shot accuracy information (e.g., fairway hit rate and hazard escape success rate), learned from past play results of the player. In an embodiment, the club-specific distance information, shot trajectory information, and shot accuracy information included in the player record information may further include information based on temperature or humidity. In an embodiment, the DB 300 may store information about the golf course. The information about the golf course may include terrain information on the golf course, par for each hole, slope of the green, pin position for each hole, and turf information on the golf course. In an embodiment, the DB 300 may store data received from the server 200 or transmit data to the server 200. For example, the DB 300 may be implemented as a non-volatile memory device, such as a Solid State Drive (SSD), for storing data.

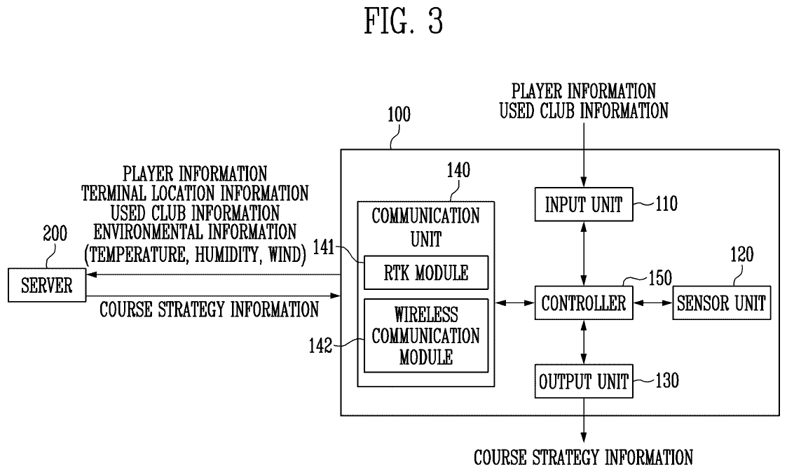

FIG. 3 is a diagram illustrating a configuration of the terminal 100 of FIG. 2.

Referring to FIG. 3, the terminal 100 may include an input unit 110, a sensor unit 120, an output unit 130, and a communication unit 140. The communication unit 140 may include a real-time kinematic (RTK) module 141 and a wireless communication module 142.

The input unit 110 may receive input from the player. The input unit 110 may include input interfaces such as a touch panel, touch screen, touch pad, touch ball, camera, microphone, gyroscope sensor, vibration sensor, or piezoelectric element.

The input unit 110 may receive player information and information about a club used (i.e., used club information) from the player. The player information may be information such as an ID and password for identifying the player. The used club information may include information about golf clubs to be used by the player during a golf round. In an embodiment, the used club information may include information about the golf club the player used to take a shot.

The sensor unit 120 may include a temperature sensor or humidity sensor for detecting environmental information (e.g., wind, temperature, humidity, and the like) of the golf course.

The output unit 130 may include output interfaces such as a Liquid Crystal Display (LCD), an Organic Light Emitting Diode (OLED) display device, an Active Matrix OLED (AMOLED) display device, an LED display device, a speaker, or a monitor.

The communication unit 140 may include the RTK module 141 and the wireless communication module 142.

The real-time kinematic (RTK) module 141 may be a GPS module to which the concept of real-time kinematic (RTK) positioning is applied, and may obtain precise positioning results in real time at a rover by using correction values for the carrier phase of a base station (GPS base) possessing precise location information. While conventional GPS modules may incur various errors due to factors like the ionosphere, atmosphere, and satellite errors, the RTK module 141 may compensate for the degradation in positioning accuracy caused by signal distortion and delays occurring when GPS satellite signals pass through the ionosphere, thereby reducing positioning errors to as little as 1 to 2 cm. In an embodiment, the RTK module 141 may receive RTK correction signals from nearby base stations using a Virtual Reference System (VRS) method, a Flachen Korrektur Parameter (FKP) method, a Master Auxiliary Concept (MAC) method, or a State Space Representation (SSR) method.

In an embodiment of the present disclosure, the location information received via the RTK module 141 may be utilized as the location information of the terminal 100. Furthermore, because the terminal 100 is mounted on the single golf cart and the single golf cart may enter the fairway and may approach very close to the ball. Therefore, in an embodiment of the present disclosure, the location of the terminal 100 acquired by the RTK module 141 may be treated as the location of the ball.

The wireless communication module 142 may connect the terminal 100 to a wireless communication network for communication with the server 200 and may be implemented in various methods, such as LTE or 5G, depending on a protocol.

A controller 150 may be a processor which controls the overall operation of the terminal 100. The controller 150 may control the input unit 110, the sensor unit 120, the output unit 130, and the communication unit 140. In an embodiment, the controller 150 may provide the player information or the used club information received via the input unit 110 to the server 200 via the wireless communication module 142 of the communication unit 140. Alternatively, the controller 150 may provide the environmental information (e.g., wind, temperature, humidity, and the like) acquired through the sensor unit 120 to the server 200 via the wireless communication module 142 of the communication unit 140. Alternatively, the controller 150 may provide terminal location information acquired via the RTK module 141 to the server 200 via the wireless communication module 142 of the communication unit 140. In various embodiments, the controller 150 may receive course strategy information from the server 200 via the wireless communication module 142 and output the course strategy information via the output unit 130 to provide the course strategy information to the player.

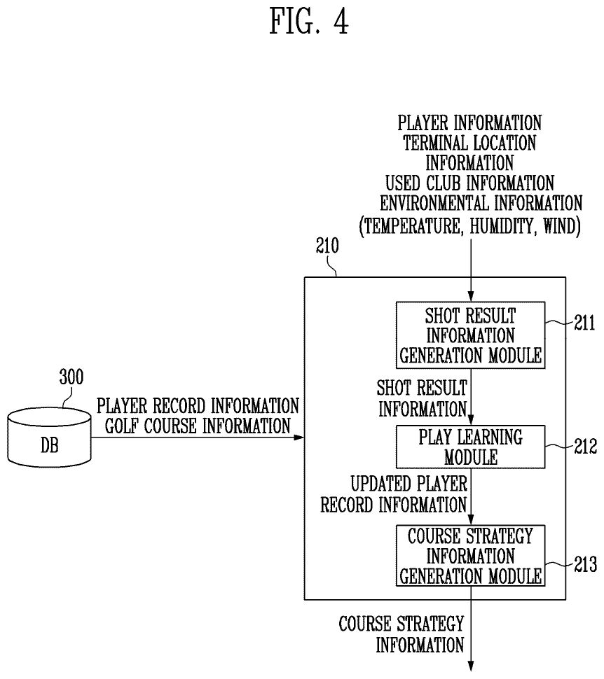

FIG. 4 is a diagram illustrating a configuration of the server 200 of FIG. 2.

Referring to FIG. 4, the AI computation module 210 of the server 200 may include a shot result information generation module 211, a play learning module 212, and a course strategy information generation module 213.

The shot result information generation module 211 may receive the player information, the terminal location information, the used club information, and the environmental information from the terminal 100. The shot result information generation module 211 may generate shot result information based on the received information.

The shot result information may include the club-specific distance information (carry and run) and shot trajectory information (pull, hook, fade, straight, draw, slice, and push).

Specifically, the shot result information generation module 211 may treat information on the distance from the previous location of the terminal to the current location of the terminal as distance information on the club indicated by the used club information. The shot result information generation module 211 may generate the shot trajectory information based on the previously-provided course strategy information and the current location information of the terminal. For example, whether the shot trajectory is a pull, hook, fade, straight, draw, slice, or push may be determined depending on how far to the left or right the current location of the terminal is offset from the provided course strategy.

The shot result information generation module 211 may provide the generated shot result information to the play learning module 212.

The play learning module 212 may acquire previously stored player record information from the DB 300. The play learning module 212 may update the player record information using the previously stored player record information and the shot result information.

In an embodiment, the player record information may include the club-specific distance information, shot trajectory information, and shot accuracy information (fairway hit rate and hazard escape success rate) learned from the past play results of the player. In an embodiment, the club-specific distance information, shot trajectory information, and shot accuracy information included in the player record information may further include information based on temperature or humidity.

The play learning module 212 may incrementally learn the club-specific distance information (carry and run), and shot trajectory information (pull, hook, fade, straight, draw, slice, and push) included in the shot result information, based on the previously stored player record information.

In an embodiment, the play learning module 212 may generate updated player record information reflecting the club-specific distance information, shot trajectory information, and shot accuracy information (fairway hit rate and hazard escape success rate) included in the player record information. In an embodiment, the play learning module 212 may either exclude records which deviate significantly from the previously stored player record information or reflect such records with a weighted value. For example, the play learning module 212 may update the club-specific distance information by averaging records excluding the highest and lowest values.

The play learning module 212 may provide the updated player record information to the course strategy information generation module 213.

The course strategy information generation module 213 may acquire golf course information from the DB 300. The course strategy information generation module 213 may generate the course strategy information using the golf course information and the updated player record information.

The course strategy information may include information about a next shot of the player which includes directional information, distance information, and an expected success rate per club. In an embodiment, the course strategy information may provide at least two types of course strategy information. For example, the course strategy information generation module 213 may provide stable course strategy information and aggressive course strategy information by using results of learning past playing tendencies of the player. The stable course strategy information may be a course strategy with a relatively high success rate, based on the club-specific distance distribution and shot trajectory distribution of the player, which utilizes the average distance and average shot trajectory. The aggressive course strategy information may be a course strategy with a relatively low success rate, based on the club-specific distance distribution and shot trajectory distribution of the player.

The course strategy information generation module 213 may provide at least two types of the generated course strategy information to the terminal 100.

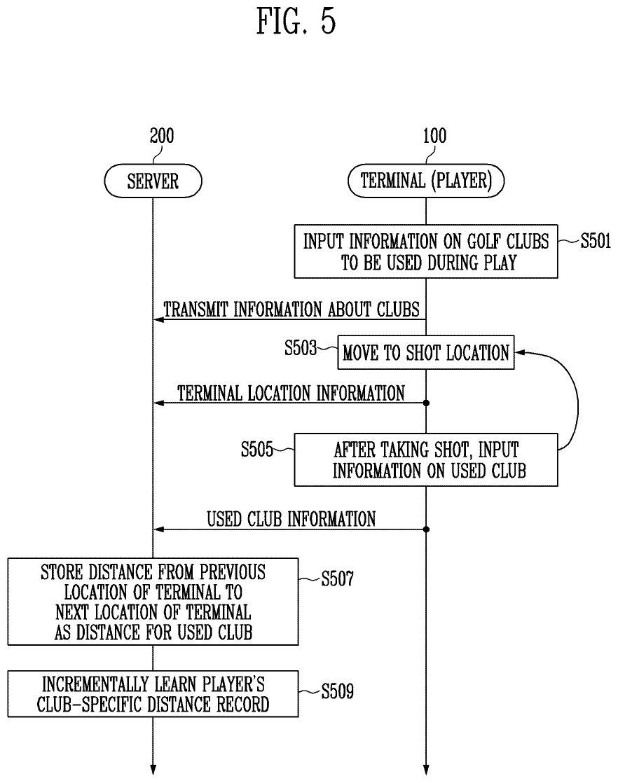

FIG. 5 is a diagram illustrating a method by which the AI caddie service provision system 1000 of the present disclosure learns the club-specific distance information of the player.

Referring to FIG. 5, in step S501, the terminal 100 may receive information from the player about the clubs to be used during play. In an embodiment, because the maximum number of clubs a player may carry for a golf game is 14, the player can input types of golf clubs they will use during play. Subsequently, the terminal 100 may transmit the information about the clubs input by the player to the server 200.

In step S503, the player may operate a single golf cart equipped with the terminal 100 to move to a shot location. Because the single golf cart may enter the fairway, the location of the terminal 100 may be treated as the location of the ball. The terminal 100 may provide terminal location information to the server 200. The terminal location information may be acquired by the RTK module 141 of the terminal 100 described with reference to FIG. 3.

In step S505, after taking a shot, the player may input information on the club used for the shot via the terminal 100. The terminal 100 may provide the used club information to the server 200.

In step S507, the server 200 may store the distance from the previous location of the terminal to the next location of the terminal as the distance for the used club.

In step S509, the server 200 may incrementally learn the distance for the used club stored in step S507 based on the player record information previously stored in the DB 300 described with reference to FIG. 4.

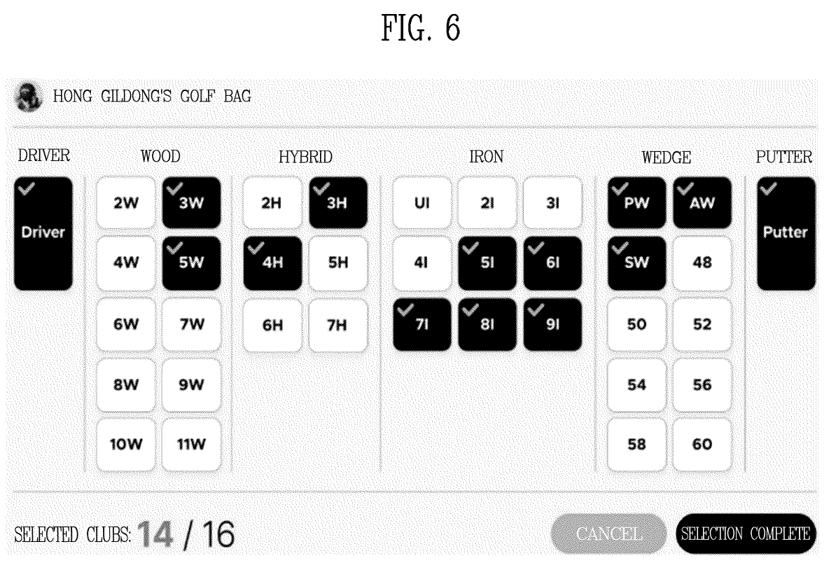

FIG. 6 is a diagram illustrating the player's input of the information about the clubs to be used in step S501 in FIG. 5.

Specifically, FIG. 6 is an example depicting a screen displayed on the output unit 130 of the terminal 100 described with reference to FIG. 3. The player may input the clubs to be used during play from among clubs included in a golf bag of the player, using a touch panel provided on the input unit 110 of the terminal 100. As shown in FIG. 6, the player may input up to 14 golf clubs to be used during play.

FIG. 6 illustrates the screen in which the player has input the golf clubs to be used during play, including a Driver, 3-wood (3W), 5-wood (5W), 3-hybrid (3H), 4-hybrid (4H), irons 5 through 9 (5I, 6I, 7I, 8I, and 9I), a Pitching Wedge (PW), Approach Wedge (AW), Sand Wedge (SW), and a Putter.

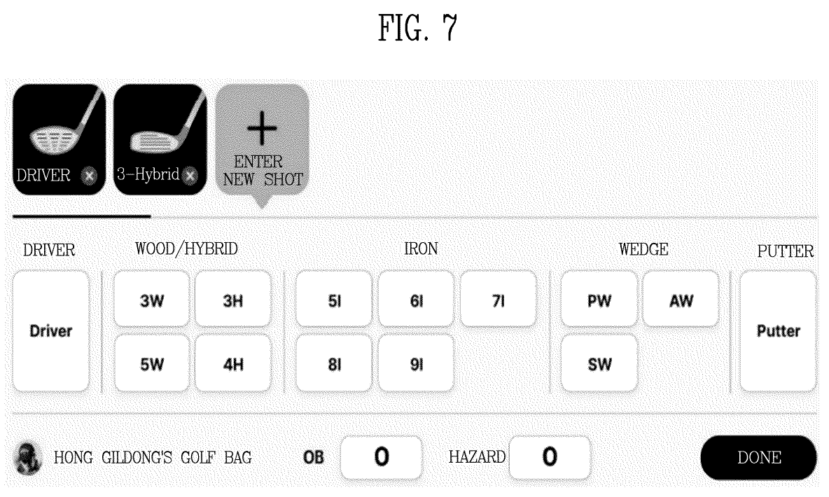

FIG. 7 is a diagram illustrating the player's input of the used club information in step S505 of FIG. 5.

After taking a shot, the player may input the type of club used for that shot via the touch panel provided on the input unit 110 of the terminal 100. Referring to FIG. 7, the player has input the following clubs to be used: a Driver, 3-wood (3W), 5-wood (5W), 3-hybrid (3H), 4-hybrid (4H), irons 5 through 9 (5I, 6I, 7I, 8I, and 9I), a Pitching Wedge (PW), Approach Wedge (AW), Sand Wedge (SW), and a Putter as shown in FIG. 6. Additionally, FIG. 7 illustrates a state in which the player has hit a tee shot with the Driver and a second shot with the 3-hybrid. The player may input the used club information into the terminal 100 by touching an “enter new shot” button, entering the information on the club used for a third shot from among the selected clubs, and then touching a “done” button.

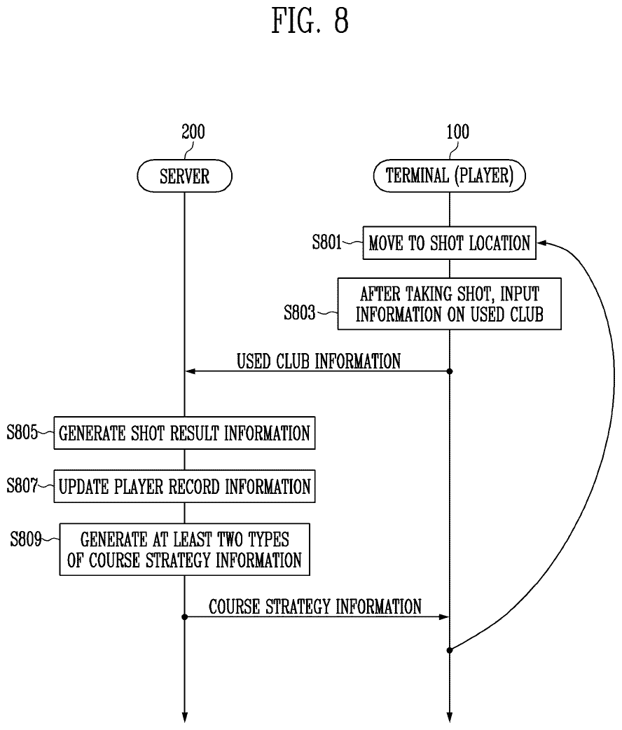

FIG. 8 is a diagram illustrating a method by which the AI caddie service provision system 1000 of the present disclosure provides the course strategy information.

Referring to FIG. 8, in step S801, the player may move to the shot location. The player may operate the single golf cart equipped with the terminal 100 to move to the shot location. Because the single golf cart may enter the fairway, the location of the terminal 100 may be treated as the location of the ball. The terminal location information may be acquired by the RTK module 141 of the terminal 100 described with reference to FIG. 3.

In step S803, after taking a shot, the player may input the information on the club used for the shot via the terminal 100. Subsequently, the terminal 100 may provide the terminal location information and used club information to the server 200.

In step S805, the server 200 may generate shot result information. The server 200 may generate the shot result information based on the terminal location information and used club information received from the terminal 100. In an embodiment, the server 200 may also receive environmental information from the terminal 100 in addition to the terminal location information and used club information.

In step S807, the server 200 may update the previously stored player record information based on the shot result information generated in step S805. Specifically, the server 200 may incrementally learn the club-specific distance information (carry and run), and shot trajectory information (pull, hook, fade, straight, draw, slice, and push) included in the shot result information, based on the previously stored player record information.

In step S809, the server 200 may generate course strategy information about a next shot of the player. In an embodiment, the course strategy information may include directional information, distance information, and information regarding an expected success rate per club for the next shot. The course strategy information may provide both stable course strategy information and aggressive course strategy information. Subsequently, the server 200 transmits at least two types of the generated course strategy information to the terminal 100, thereby enabling the course strategy information to be provided to the player.

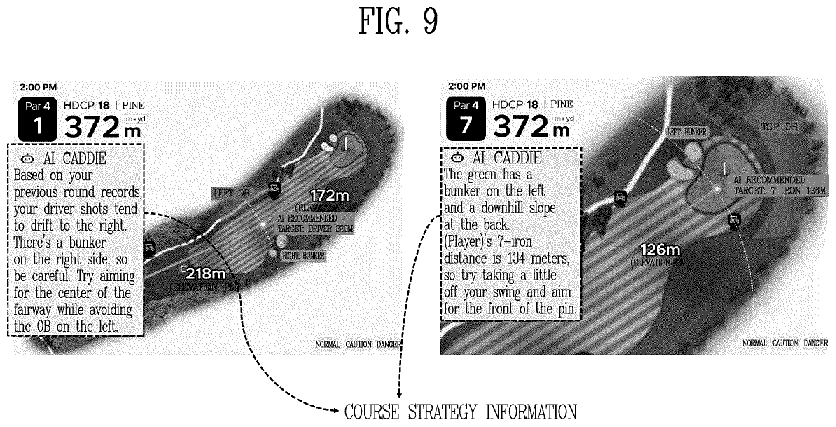

FIG. 9 is a diagram illustrating an example of an AI caddie service provided by the AI caddie service provision system 1000 of the present disclosure.

Referring to FIG. 9, the output unit 130 of the terminal 100, described with reference to FIG. 3, may provide the AI caddie service to the player. That is, the course strategy information provided from the server 200 may be provided to the player as player-specific information in the form of an “AI caddie,” as illustrated in FIG. 9. The AI caddie service may provide optimal advice for a shot situation at each hole based on previous round records of the player and shot trajectory information. Specifically, the AI caddie service may provide information such as which club is the appropriate choice based on the club-specific distance information of the player, and which terrain features to aim at for advantageous shots. However, the “AI caddie” illustrated in FIG. 9 is a mere example of an embodiment of the present disclosure, and based on the descriptions with reference to FIGS. 3 and 4, various advice may be provided considering factors like temperature, humidity, and the condition of the golf course's turf.

Although the present disclosure has been described with reference to specific embodiments and drawings, the present disclosure is not limited to these embodiments. Those skilled in the art to which the present invention pertains may make various modifications and variations based on the above description.

Therefore, the scope of the present disclosure should not be limited to the described embodiments, but should be defined by the appended claims and equivalents thereof.

In the above-described embodiments, all steps may be performed selectively or omitted. Furthermore, in each embodiment, the steps need not necessarily occur in sequence and may be reversed. The embodiments disclosed in this specification and the drawings are merely specific examples provided to readily explain the technical content of this specification and aid in its understanding, and are not intended to limit the scope of this specification. That is, it is obvious to those skilled in the art to which this specification pertains that other variations based on the technical spirit of this specification are feasible.

The present specification and drawings disclose preferred embodiments of the present disclosure. Although specific terms are used, they are employed in a general sense to facilitate explanation of the technical content and aid understanding of the present disclosure, and are not intended to limit the scope of the present disclosure. It is obvious to those skilled in the art to which the present invention pertains that other variations based on the technical spirit of the present invention are possible beyond the embodiments disclosed herein.

Embodiments of the present disclosure may provide an artificial intelligence (AI) caddie service provision system and an operating method thereof.

What is claimed is:

1. An artificial intelligence (AI) caddie service provision system comprising:
a terminal mounted on a single golf cart in which a player rides and receiving information on a club used for a shot after the player takes the shot and moves the single golf cart to a new location of a ball; and
a server learning distance information of the club used for the shot based on location information of the terminal at the new location of the ball and the information on the club used for the shot, and then providing the terminal with strategy information for a next shot to be taken from the new location of the ball.

2. The AI caddie service provision system of claim 1, wherein the terminal comprises:
a real-time kinematic (RTK) module acquiring the location information of the terminal using RTK positioning;
a wireless communication module connected to a wireless communication network to perform data communication between the terminal and the server; and
a controller controlling the wireless communication module to provide the location information of the terminal to the server.

3. The AI caddie service provision system of claim 2, further comprising a database storing player record information which includes previous play information of the player and information on a golf course on which the player plays a round.

4. The AI caddie service provision system of claim 3, wherein the server comprises:
a shot result information generation module receiving the location information of the terminal and the information on the club used for the shot from the terminal, and generating shot result information including club-specific distance information and shot trajectory information of the player;
a play learning module acquiring the player record information from the database and updating the player record information based on the shot result information; and
a course strategy information generation module generating course strategy information for the next shot based on the updated player record information.

5. The AI caddie service provision system of claim 4, wherein the shot result information generation module determines information on a distance from a location of the terminal from which the player took the shot to the location of the terminal at the new location of the ball as the distance information of the club used for the shot.

6. The AI caddie service provision system of claim 5, wherein the player record information includes club-specific distance information, shot trajectory information, and shot accuracy information learned from past play results of the player.

7. The AI caddie service provision system of claim 6, wherein the learned club-specific distance information, shot trajectory information, and shot accuracy information included in the player record information further include information based on temperature or humidity.

8. The AI caddie service provision system of claim 7, wherein the course strategy information generation module generates stable course strategy information and aggressive course strategy information by using results of learning past playing tendencies of the player.

9. The AI caddie service provision system of claim 7, wherein the course strategy information includes directional information, distance information, and an expected success rate per club for the next shot.

10. The AI caddie service provision system of claim 1, wherein the single golf cart is capable of directly entering a fairway included in a golf course and moving to a location where the ball of the player is located, except for a pre-set restricted area.
